(19) United States
(12) Patent Application Publication
(10) Pub. No.: US 2020/0069357 A1
(43) Pub. Date: Mar. 5, 2020

(54) ELECTROSURGICAL GENERATOR VERIFICATION SYSTEM

(71) Applicant: Applied Medical Resources Corporation, Rancho Santa Margarita, CA (US)

(72) Inventors: Sean Adl, Rancho Santa Margarita, CA (US); Andrew Soto, Rancho Santa Margarita, CA (US); Igor Gorin, Rancho Santa Margarita, CA (US)

(21) Appl. No.: 16/562,122

(22) Filed: Sep. 5, 2019

Related U.S. Application Data
(60) Provisional application No. 62/727,176, filed on Sep. 5, 2018.
(60) Provisional application No. 62/727,195, filed on Sep. 5, 2018.

(51) Int. Cl. A61B 18/12; A61B 18/14; G16H 20/40; G16H 40/40

(52) U.S. Cl. CPC A61B 18/1206; A61B 18/1445; A61B 2018/00875; G16H 40/40; G16H 20/40

(57) ABSTRACT

Systems and methods for performing a self-verification system test upon activation of an electrosurgical generator are described. The systems and methods allow for enhancing surgical outcomes by providing generators having accurate RF energy generation, measurement, calibration and self-testing system. This is achieved through implementation of an automated self-verification process at a power start-up of the generator, which allows for rapidly identifying a potential generator issue prior to any use of a connected electrosurgical instrument or supply of any RF energy to the tissue or vessel through the electrosurgical instrument. Additionally, one or more internal impedance loads are integrated within the electrosurgical generator. The internal impedance loads with multiple configurations are utilized to verify the voltage, current, power, and/or phase measurements of the generator. By incorporating or integrating the self-verification process and its related hardware resources within the electrosurgical generator, many improvements in outcome of pre-surgical procedures may be achieved.

### CROSS-REFERENCE TO RELATED APPLICATIONS

This application claims the benefit of both co-pending U.S. Provisional Application Ser. No. 62/727,176 filed on Sep. 5, 2018, and U.S. Provisional Application Ser. No. 62/727,195 filed on Sep. 5, 2018, which are hereby expressly incorporated by reference in their entirety for all purposes.

### TECHNICAL FIELD

The present disclosure is generally directed to electrosurgical generator systems and methods and more particularly to output verification of electrosurgical generators configured to supply radiofrequency (RF) energy.

### BACKGROUND

Electrosurgical devices or instruments have become available that use radiofrequency (RF) energy to perform certain surgical tasks such as coagulate, fuse, or cut tissue. Such electrosurgical instruments typically fall within two classifications: monopolar and bipolar. In monopolar instruments, electrical energy is supplied to one or more electrodes on the instrument with high current density while a separate return electrode is electrically coupled to a patient and is often designed to minimize current density. Bipolar electrosurgical instruments, which operate without separate return electrodes, can deliver electrical signals to a focused tissue area with reduced risks.

Even with the relatively focused surgical effects of bipolar electrosurgical instruments, surgical outcomes are often highly dependent on surgeon skill. Enhanced generators have been made to reduce this dependency. However, such generators have had shortcomings in that they can provide inconsistent results in determining tissue coagulation, fusion, or cutting endpoints for varied tissue types or combined tissue masses. These systems can also fail to provide consistent electrosurgical results among use of different instruments; having different instrument and electrode geometries and subjected to different tissue types and tissue amounts. Some of these shortcomings are sometimes exacerbated by inaccurate RF energy generation, measurement, calibration and testing of such systems. Unavailable or unreliable test equipment and/or unavailable or inexperienced personnel also contribute to these shortcomings or do little to assist in overcoming these shortcomings. Hence, embodiments of the present invention are intended to obviate or at least alleviate the aforementioned problems of the conventional electrosurgical generators.

### SUMMARY

In accordance with various embodiments, an electrosurgical system for sealing, fusing and/or cutting tissue is provided. The electrosurgical system comprises an electrosurgical generator and an electrosurgical instrument or device. The electrosurgical generator in various embodiments may include an RF amplifier, a feedback system and a primary controller. The RF amplifier supplies RF energy through a removably coupled electrosurgical instrument configured to fuse, seal and/or cut tissue. The primary controller is arranged to initiate and halt the supply of RF energy and the feedback system is arranged to monitor the supplied RF energy. By monitoring or verifying the electrical properties of the RF output, the electrosurgical generator of the present invention, in accordance with various embodiments, ensures the RF energy being outputted is optimal for surgical treatment of tissue or vessels.

In accordance with one aspect of the present invention, an electrosurgical system for performing surgical procedures is provided. The electrosurgical system may include an electrosurgical generator adapted to perform a self-verification system test upon activation of the generator and a plurality of internal impedance loads. The electrosurgical generator includes a processor which is configured to cause initiating the self-verification system test after determining a predetermined period of time has elapsed after starting the generator. If a failure of the generator is not detected, the processor allows for supplying RF energy to a connected electrosurgical hand device. If a failure of the generator is detected by the self-verification system test for a number of consecutive times, the processor generates a system error and notifies the user or surgeon of the generator's error.

In accordance with a second aspect of the present invention, a method for performing an auto-verification and self-verification system of an electrosurgical generator prior to performing surgical procedure is provided. The method includes the steps of: initiating a self-verification system test after determining a predetermined period of time has elapsed upon activation of the generator; setting RF regulation modes and RF resolution settings after initiating the self-verification system test; generating RF energy and directing RF output to a plurality of impedance loads within the generator; determining whether the self-verification system test has been completed; and recording self-verification system test completion timestamp upon completion of the self-verification system test. After completion of the self-verification system test, the method further includes a step of determining whether a generator failure has occurred, and accordingly, allows for initiating or halting the supply of RF energy to a connected electrosurgical instrument.

In accordance with a third aspect of the present invention, a control system for use with an electrosurgical generator adapted to perform a self-verification system test upon activation of the generator is provided. The control system may include an RF amplifier for supplying RF energy, a feedback system for continually monitoring electrical properties of the supplied RF energy and generating digital RF signals relating thereto and a primary microcontroller configured to initiate the self-verification system test and to allow supplying and halting the supplied RF energy to a connected electrosurgical hand device based at least in part on results of the self-verification system test.

In accordance with a fourth aspect of the present invention, there is provided an electrosurgical system for performing surgical procedures. The electrosurgical system may include an electrosurgical generator adapted to supply electrosurgical RF energy to tissue and an electrosurgical instrument comprising at least one active electrode adapted to apply the supplied electrosurgical RF energy to tissue. The electrosurgical generator may include a microprocessor programmed to regulate the RF output of the generator to a predetermined RF regulation value across a plurality of RF regulation modes and a plurality of RF resolution settings. The electrosurgical generator further includes a feedback system configured to measure electrical properties of the RF output supplied to one or more internal impedance loads across a plurality of channels. The microprocessor is further programmed to compare all measurements values of the feedback system channels for each of the plurality of RF regulation modes and RF resolution settings and determine whether a fault condition exist within the generator.

In accordance with a fifth aspect of the present invention, an electrosurgical generator is provided. The electrosurgical generator may include a plurality of impedance loads integrated within an RF amplifier that supplies RF energy and a feedback system measuring the electrical properties of RF energy directed to the plurality of impedance loads across a plurality of channels. The electrosurgical generator further includes a primary microcontroller configured to initiate a self-verification system test upon activation of the generator to verify the RF output of the generator across a plurality of RF regulation modes and RF resolution setting for determining whether a generator failure exist.

In accordance with various embodiments, an electrosurgical generator is provided. The generator comprises a plurality of switchable impedance loads integrated within the generator and through which RF energy is supplied there through and a microcontroller configured to switch in one or more of the plurality of switchable impedance loads to verify RF output of the generator.

Many of the attendant features of the present inventions will be more readily appreciated as the same becomes better understood by reference to the foregoing and following description and considered in connection with the accompanying drawings.

### BRIEF DESCRIPTION OF THE DRAWING

The present disclosure is described in conjunction with the appended figures:

FIG. 1 is a perspective view of an electrosurgical generator in accordance with various embodiments of the present invention.

FIG. 2 is a perspective view of an electrosurgical hand device in accordance with various embodiments of the present invention.

FIG. 3 is a perspective view of an alternative embodiment of an electrosurgical hand device in accordance with various embodiments of the present invention.

FIG. 4 depicts a block diagram of an electrosurgical generator in accordance with various embodiments of the present invention.

FIG. 5 depicts a block diagram of an embodiment of a control system of an electrosurgical generator coupled to an electrosurgical hand device.

FIG. 6 depicts, in greater detail, a block diagram of an embodiment of a feedback system within a control system of an electrosurgical generator.

FIG. 7 is a schematic illustration of an example of hardware resources implemented within an RF amplifier of an electrosurgical generator to perform self-verification system test.

FIGS. 8-9 illustrate flow diagrams of various embodiments of a self-verification system operations or processes in accordance with various embodiments of the present invention.

FIG. 10 illustrates a flow diagram of an example method for performing a self-verification system test of an electrosurgical generator.

In the appended figures, similar components and/or features may have the same reference label. Where the reference label is used in the specification, the description is applicable to any one of the similar components having the same reference label.

### DETAILED DESCRIPTION

The ensuing description provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiments of the disclosure. It should be understood that various changes may be made in the function and arrangement of elements without departing from the spirit and scope of the invention as set forth in the appended claims.

This disclosure relates in general to electrosurgical systems. It specifically relates to a new generation of electrosurgical generators capable of initiating and performing an auto-verification and self-verification system test of the generators.

To maintain an electrosurgical generator, verification of the generator's output is often required to ensure the RF being outputted from the generator is as intended. This verification can be performed periodically, e.g., every twelve months or two years and often by a user interactive or manual system or process. The system or process may also require the connection of various external devices to the generator. These external devices may include a verification adapter, external loads, e.g., 10-500 Ohm loads with decreasing power ratings and increasing voltage ratings, and/or measuring devices, e.g., oscilloscope, differential voltage probe and current probe.

The user must then connect the appropriate adapter, loads and/or measuring devices and perform specified tests requiring adjustments to the generator, external devices and/or measuring devices and connections thereto. Additionally, appropriate monitoring and recording the test results are required along with understanding errors and physically removing a faulty generator from use. Also, to perform the generator's output verification, the generator must be taken out of circulation and not be used in a surgical procedure, and often times not performed at a surgical site such as, for example, hospitals, due to lack of experience, personnel or equipment.

Embodiments of the present invention are directed to systems and methods for enhancing surgical outcomes by providing generators having accurate RF energy generation, measurement, calibration and/or self-testing system. The present invention in accordance with various embodiments allows detection of failure upon activation, thereby reducing troubleshooting and surgical operational time while avoiding other potential surgical difficulties. This is achieved through implementation of an automated self-verification process at a power start-up of the electrosurgical generator, which allows for rapidly identifying a potential generator issue prior to any use of a connected electrosurgical instrument or supply of any RF energy to the tissue or vessel through the electrosurgical instrument.

The electrosurgical generators in accordance with various embodiments may include one or more internal impedance load for performing the self-verification process. The internal impedance load with multiple configurations are utilized to verify the voltage, current, power, and/or phase measurements of the generator. By incorporating or integrating the self-verification process and its related hardware resources within the electrosurgical generator, many improvements in outcome of pre-surgical procedures may be achieved. By way of examples, these improvements may include, but not limited to, elimination for connecting external devices to the generator, thereby removing the risk of using unavailable or unreliable test equipment, and/or unavailable or inexperienced personnel. In addition, the need for taking the generator out of circulation for performing the generator's output verification is completely eliminated.

In the following, the electrosurgical system and method according to various embodiments of the present invention is explained in detail with sections individually describing: the electrosurgical generator, the electrosurgical instrument and the control system and method used for performing the automated self-verification system test of the generator.

In accordance with various embodiments, an electrosurgical generator is provided that controls the delivery of electrosurgical or radiofrequency (RF) energy, adjusts the RF energy and in various embodiments measures and monitors electrical properties, e.g., phase, current, voltage and/or power, of the supplied RF energy to a connectable electrosurgical instrument to ensure optimal sealing, fusing and/or cutting of tissues or vessels. In various embodiments, the generator may include a feedback system that determines such electrical properties and through a microcontroller regulates and/or controls an RF amplifier that generates the required RF energy to provide the optimal RF output for sealing, fusing and/or cutting tissue/vessels under dynamic conditions, such as for example, varying loads, procedural or operational conditions.

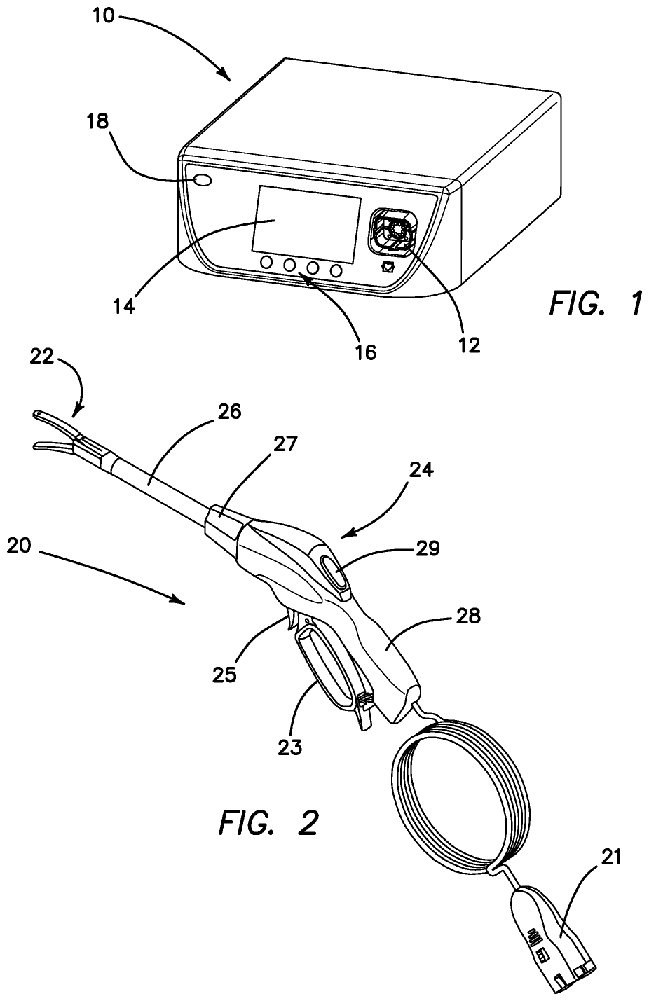

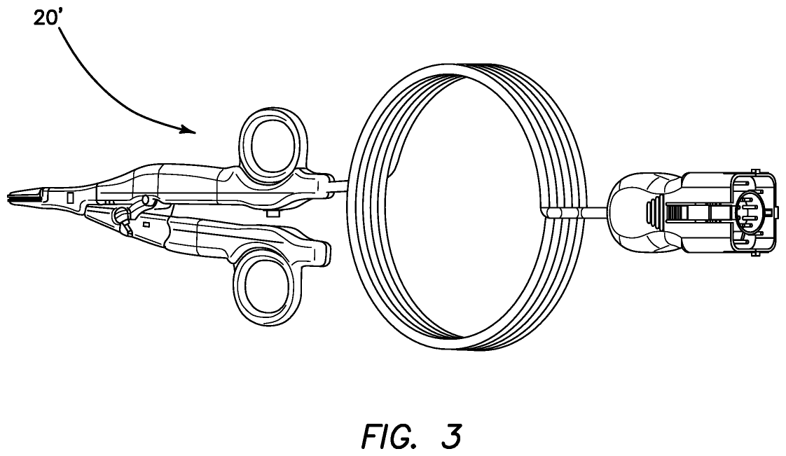

Referring first to FIGS. 1-2, an exemplary embodiment of an electrosurgical system according to various embodiments of the present invention is illustrated. As shown in these figures, the electrosurgical system may include an electrosurgical generator 10 and a removably connectable electrosurgical tool or instrument 20. The electrosurgical hand device or instrument 20 can be electrically coupled to the generator 10 via a cabled connection with a device key or connector 21 extending from the instrument 20 to a device connector or access port 12 on the generator 10. The electrosurgical instrument 20 may include audio, tactile and/or visual indicators to apprise a user of a particular or predetermined status of the instrument 20 such as, for example, a start and/or end of a fusion operation. In some embodiments, a manual controller such as a hand or foot switch can be connectable to the generator 10 and/or instrument 20 to allow predetermined selective control of the instrument such as to commence a fusion operation.

In accordance with various embodiments, the electrosurgical generator 10 includes a display 14 that may indicate the status of the electrosurgical system including, among other information, the status of the one or more electrosurgical instruments and/or accessories, connectors or connections thereto, the state or operations of the generator and error indicators. The electrosurgical generator 10 in accordance with various embodiments of the present invention may include a user interface such as, for example, a plurality of buttons 16. The plurality of buttons 16 allows for user interaction with the electrosurgical generator 10. This user interaction may include, for example, requesting an increase or decrease in the electrical energy supplied to one or more instruments 20 that are coupled to the electrosurgical generator 10. In various embodiments, the generator 10 further includes a user-accessible power-on switch or button 18 that when activated powers the generator 10 and activates or initiates a self-verification system test of the generator. In other embodiments, the display 14 can be a touch screen display thus integrating data display and user interface functionalities.

In various embodiments, the electrosurgical generator 10 of the present invention is configured to output radiofrequency (RF) energy through the connectable electrosurgical instrument or hand device 20 to seal, fuse and/or cut tissue or vessels via one or more electrodes. The electrosurgical generator 10, according to the embodiments of the present invention, is configured to generate up to 300V, 8 A, and 375VA of RF energy and it is also configured to determine a phase angle or difference between RF output voltage and RF output current of the generator during activation or supply of RF energy. In this way, the electrosurgical generator 10 regulates voltage, current and/or power and monitors RF energy output (e.g., voltage, current, power and/or phase). In one embodiment, the generator 10 may stop, terminate or otherwise disrupt RF energy output under predetermined conditions. By way of example, these predetermined conditions may be any of the following conditions: when a device switch is de-asserted (e.g., fuse button released), a time value is met, and/or active phase angle and/or change of phase is greater than or equal to a phase and/or change of phase stop value indicating end of an operation such as fusion or cutting of tissue.

The electrosurgical instrument 20, according to the embodiments of the present invention, may include an elongate shaft 26 having a proximal end coupled to or from which an actuator 24 extends and a distal end coupled to or from which jaws 22 extend. A longitudinal axis extending from the proximal end to the distal end of the elongate shaft 26. In one embodiment, the actuator 24 may include a movable handle 23 which is pivotably coupled to a stationary handle or housing 28. The movable handle 23 is coupled to the stationary handle or housing 28 through a central or main floating pivot. In operation, the movable handle 23 is manipulated by a user, e.g., a surgeon, to actuate the jaws 22 at the distal end of the elongate shaft 26, and thereby, selectively opening and closing the jaws 22. When tissue or vessels are grasped between the jaws 22, a switch or button 29 is activated by the surgeon to seal, fuse and/or cut the tissue/vessels between the jaws 22. Once the button 29 is activated, associated circuitry or contacts are connected to connect appropriate electrodes of the jaws with associated connections of the generator 10 to supply RF energy to tissue grasped between the jaws 22 or otherwise in contact with the one or more electrodes of the jaws.

In various embodiments, the electrosurgical instrument 20 further includes a mechanical or electrical cutting blade that can be coupled to a blade actuator such as a blade lever or trigger 25 of the stationary handle or housing 28. The cutting blade is actuated by the blade trigger 25 to divide or cut the tissue between the jaws 22. In various embodiments, a blade slider is connected to the blade trigger 25 and a protrusion extends from a proximal portion of the blade slider into an opening in one end of the blade trigger connecting the components together. The other end of the blade trigger is exposed and accessible by the user with the blade trigger 25 being pivotable about a trigger pivot at or near the mid-point of the blade trigger. As such, as the blade trigger 25 is pulled or rotated by the user proximally, the end of the blade trigger connected to the blade slider slides or moves the blade slider distally. Integrated with or attached to a distal end of the blade slider is a cutting blade, knife or cutting edge or surface. As such, as the blade slider translates longitudinally through a blade channel in the jaws, tissue grasped between the jaws 22 is cut. In one embodiment, the cutting edge or surface is angled to facilitate cutting of the tissue between the jaws 22. In various embodiments, the cutting blade is a curved blade, a hook, a knife, or other cutting element that is sized and configured to cut tissue between the jaws 22.

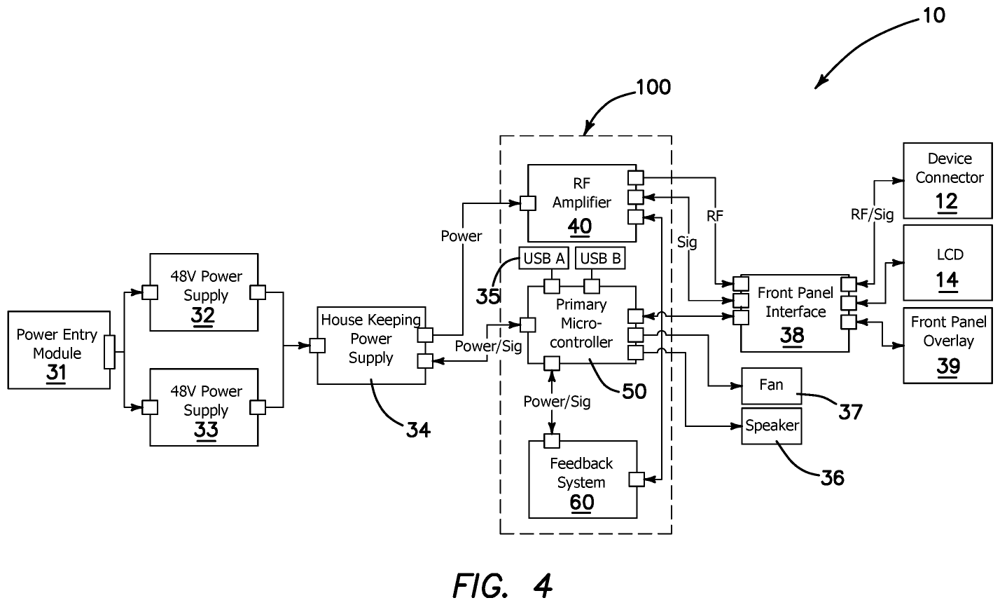

In accordance with various embodiments, the elongate shaft 26 comprises an actuation tube or rod coupling the jaws 22 with the actuator. In one embodiment, the actuator includes a rotation shaft assembly including a rotation knob 27 which is disposed on an outer cover tube of the elongate shaft 26. The rotation knob 27 allows a surgeon to rotate the shaft of the device while gripping the actuator. In various embodiments, the elongate shaft 26 is rotatable 360 degrees and in other embodiments, rotation of the elongate shaft 26 is limited to 180 degrees, i.e., ninety degrees clockwise and ninety degrees counter clockwise. FIG. 3 illustrates an alternative embodiment of an electrosurgical hand device 20′ connectable to the electrosurgical generator 10. The electrosurgical hand device 20′ is similar but includes different features and has a different surgical use than the electrosurgical hand device 20.

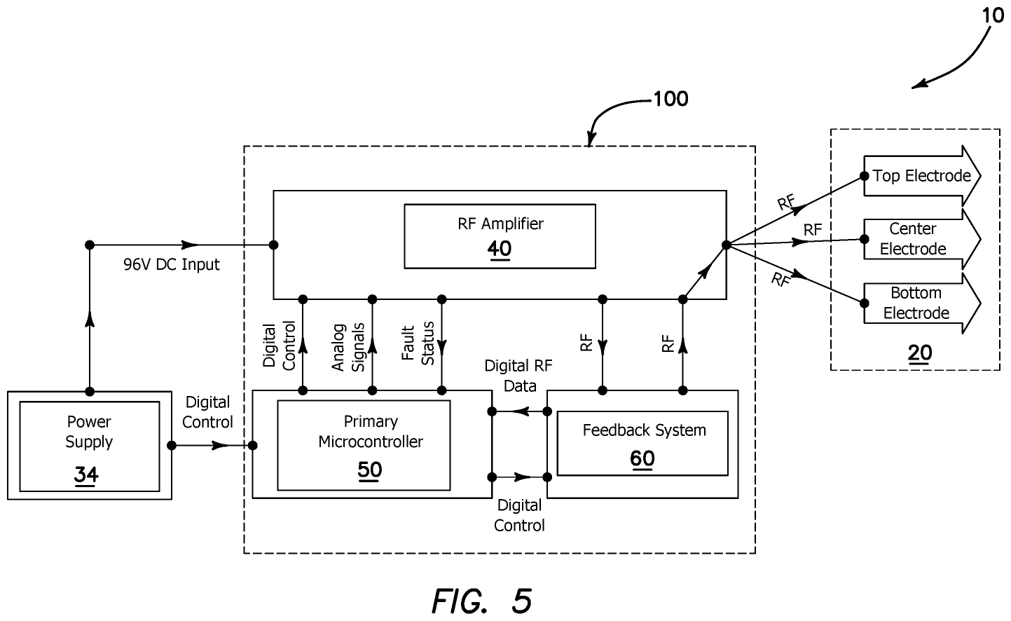

Referring next to FIG. 4, a block diagram of an electrosurgical generator 10 according to the embodiments of the present invention is shown. As shown in this figure, the electrosurgical generator 10 may include a power entry module 31, e.g., an AC main input, coupled to a power supply module, e.g., two 48V DC power supplies 32, 33. The power supply module converts the AC voltage from the AC main input to a DC voltage and via a house keeping power supply 34 provides power to various circuitry of the generator 10 and in particular supplies power to an RF amplifier 40 that generates or outputs the RF energy. In one embodiment, the RF amplifier 40 may include a combined Buck and H-Bridge circuitry to convert a DC voltage input into an RF output and in another embodiment into a variable amplitude 350 kHz sine wave. The DC voltage input is a 96V DC input that is generated by the two 48V DC power supplies 32, 33 coupled in series. One of the 48V DC power supply 32, 33 is configured to generate low voltage rails and in particular supply standby voltage to power on the generator 10.

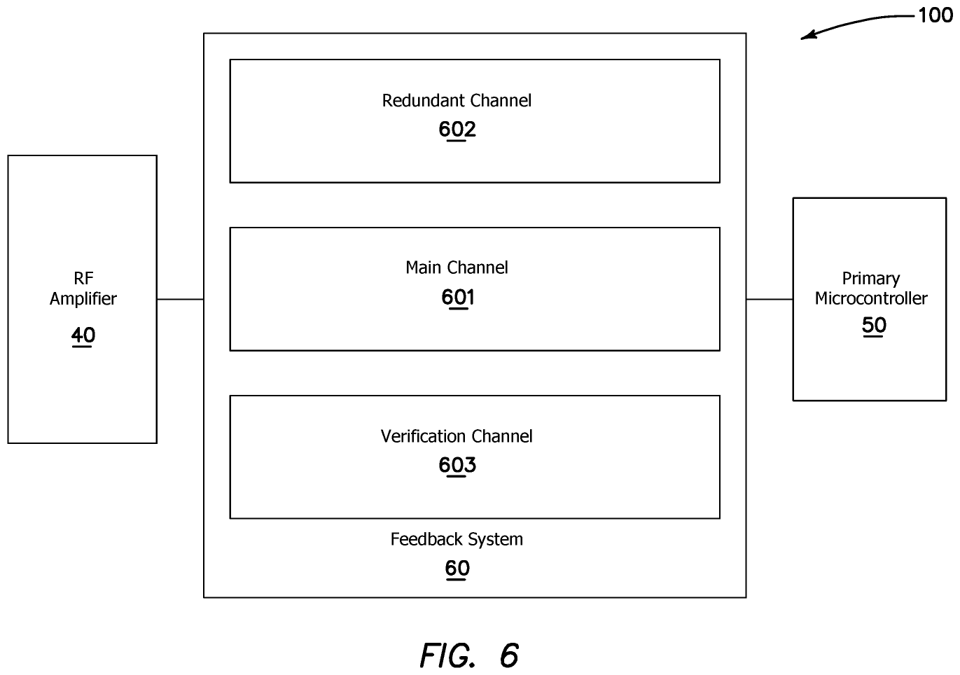

The RF output and in various embodiments the amplitude of the RF waveform output is controlled and regulated by an electrosurgical control system or a digital integral servo control system 100 embedded or integrated within the electrosurgical generator 10. FIG. 5 illustrates, in greater detail, a block diagram of an embodiment of a control system 100 of the electrosurgical generator 10 coupled to an electrosurgical hand device 20. As shown in FIGS. 4-5, the control system 100 may include the RF Amplifier 40, a primary microcontroller 50 and a feedback system 60. The control system 100 varies between regulating voltage, current, or power of the RF output generated by the RF Amplifier 40. In various embodiments, the feedback system 60 measures the RF output and, after processing the measured data, digitally feeds the RF output's real and imaginary components to the primary microcontroller 50. The primary microcontroller 50, according to the embodiments of the present invention, may include a primary field programmable gate array (FPGA) and a processor. By way of example, the processor of the microcontroller 50 may include an advanced reduced instruction set machine (ARM) processor. The primary FPGA processes the received data from the feedback system 60 and adjusts the output of the RF amplifier 40 to meet a desired regulation target. In various embodiments, the feedback system 60 comprises of analog input, digital processing and digital output.

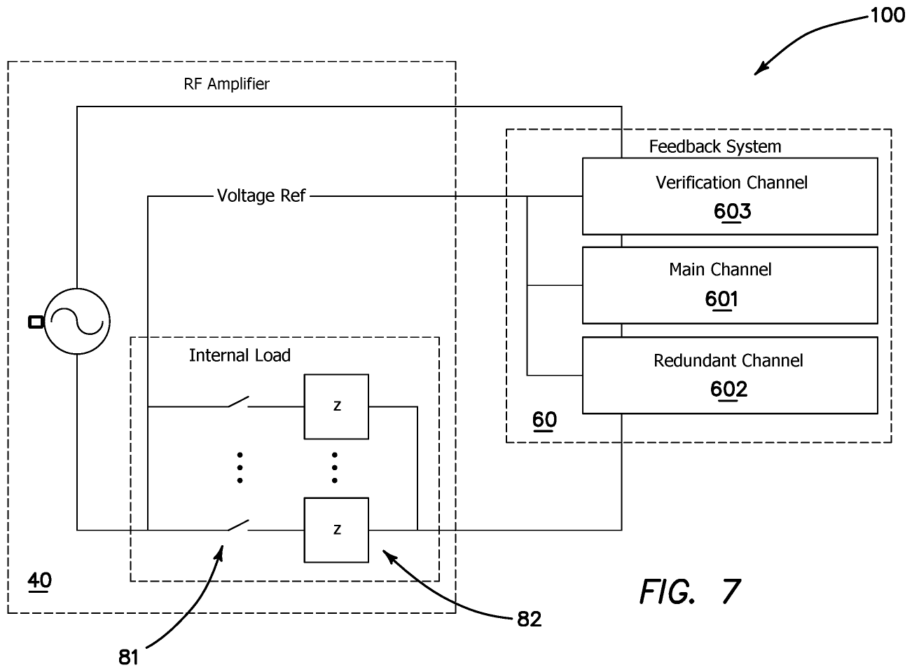

With reference to FIG. 6, a block diagram of an embodiment of the feedback system 60 within the control system 100 of the electrosurgical generator 10 is shown in greater detail. As shown in this figure and in accordance with various embodiments of the present invention, the verification system 60 may include a main channel 601 and a redundant channel 602. The main channel 601 and redundant channel 602 in various embodiments may include separate but identical components. Additionally, the main and redundant channels 601 and 602 follow separate but identical electrical paths and in one embodiment are both connected to the RF amplifier 40 and the RF output.

The feedback system 60 further includes a verification channel 603 which in various embodiments is separate but similar to the main channel 601 and redundant channel 602. The verification channel 603 may include components that are separate from the other channels but are similar. In one embodiment, the verification channel 603 may include the same components as the main and redundant channels 601 and 602, but the components in the verification channel 603 have higher ratings, e.g., higher resolution and/or lower drift, and are often more costly. In another embodiment, the verification channel 603 may include the same components as the main and redundant channels 601 and 602. However, in various embodiments, the verification channel 603 is only used in the operation of or by the self-verification test system. As such, the components of the verification channel 603 are used less often and thus may remain more accurate, e.g., have less drift, than the components of the main and/or redundant channels 601 and 602 that are constantly used throughout the generator's operations. In various embodiments, the verification channel 603 follows a separate but identical electrical path as the main and redundant channels 601 and 602 and in one embodiment is connected to the RF amplifier 40 and the RF output.

The channels 601, 602 and/or 603 in accordance with various embodiments comprises circuitry including active and/or passive components and electrical pathways connected thereto arranged to transmit and/or measure the RF energy supplied from the RF amplifier to the microcontroller and/or as directed by the microcontroller. In accordance with various embodiments, one or more or all the channels includes and/or are connected to one or more measurement circuitry comprising sensors, detectors, comparators, resistors, memory and/or the like and/or any combination thereof configured to measure, calculate and/or record such measurements of electrical properties (e.g., current, voltage, power, and/or phase) of the RF energy directed across the one or more or all the channels.

In accordance with various embodiments of the present invention, the electrosurgical generator 10 is further configured to provide RF output in three resolution settings: low voltage, normal or medium voltage and high voltage ranges. In various embodiments, device scripts stored and located on connectable electrosurgical hand devices, e.g., instrument 20, and/or connectors coupled thereto, e.g., device key 21, are used to determine or set the RF output or voltage mode.

In various embodiments, the electrosurgical generator 10 logs all RF output data onto an internal memory device, e.g., a secure digital (SD) or non-volatile memory card. The memory device is configured to be read through an interface port 35, e.g., a universal serial bus (USB) port, on the electrosurgical generator 10 (best shown in FIG. 4). In various embodiments, the generator 10 is configured to copy the data from the internal memory device to a connectable portable storage device, e.g., a USB flash drive, through the interface port of the generator.

Reffering back to FIGS. 1 & 4-5 and in accordance with various embodiments, the electrosurgical generator 10 is configured to alert the surgeon when the vessel has reached a completed procedure state, e.g., a completed seal state, or if an error or fault condition has occurred. The electrosurgical generator 10 in various embodiments may include visual, tactile and/or audible outputs to provide such alerts or other indicators or information to the surgeon as dictated by the surgical procedure, device script or health or operational information regarding the device 20 and/or generator 10. In one embodiment, the generator 10 via a front panel interface 38 alerts the surgeon through the LCD display 14, which is integrated into a front panel of the generator, and in various embodiments provides specific audible alarm or informational tones through a speaker 36 also integrated into the front panel of the generator. The generator 10 in various embodiments may include a front panel overlay 39 that provides a user interface or access including navigational push buttons to allow user access to systems settings such as volume or display brightness. The front panel overlay 39 may also include the system power button or connection. In various embodiments, a fan system 37 is provided to assist in heat dissipation. Additionally, as illustrated in the FIGS. 4-5, signal or sig represents connections that, for example, comprise of digital signals used to communicate information across systems and/or printed circuit boards, power represents connections that, for example, comprise of voltage rails used to power systems and/or printed circuit boards and RF represents connections that, for example, comprise of high voltage, high current RF energy used to seal, fuse or cut tissue or vessels.

As described further above, the electrosurgical generator 10 in various embodiments further includes the user-accessible power-on switch or button 18 that is accessible by a surgeon to activate or turn on the generator 10. In one embodiment, the power-on switch 18 is on a front panel of the generator. In accordance with various embodiments, once the generator 10 is activated by the activation of the power-on switch 18, the generator 10 initiates or activates a power on self-verification system test. During the self-verification system test, in various embodiments, the generator 10 verifies regulation of the RF output in one or more RF modes and/or one or more RF resolution settings. In accordance with various embodiments, the RF regulation modes include voltage, current and power regulation modes and the RF resolution settings include low, normal and high voltage settings.

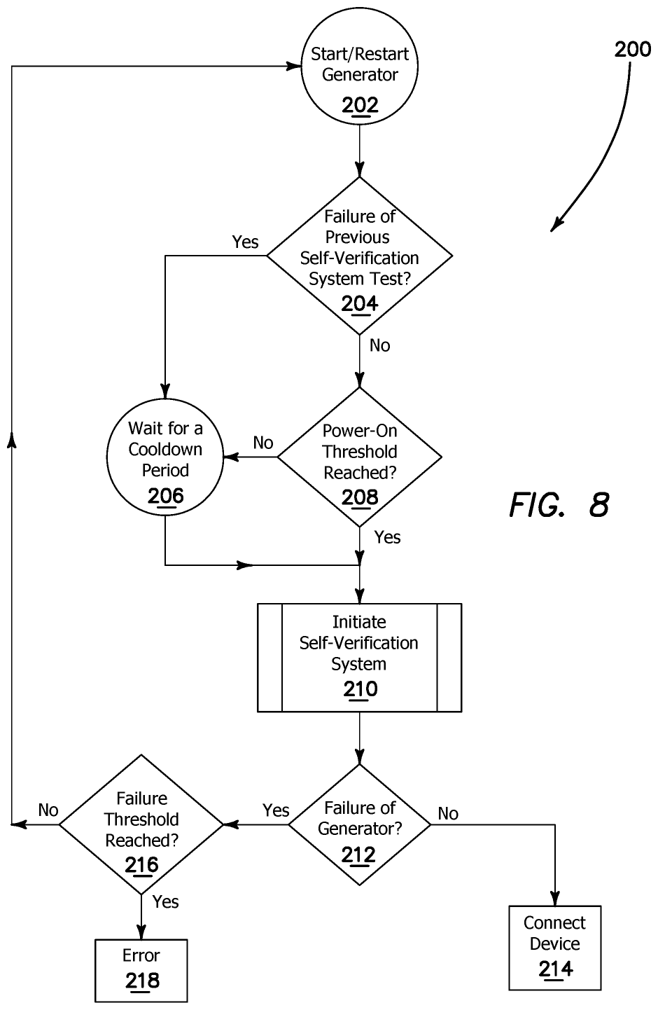

With reference to FIG. 7, a schematic illustration of an example of hardware resources implemented within an electrosurgical generator 10 to perform self-verification system test is shown. As it can be seen from this figure, one or more impedance load 82 may be implemented within the RF amplifier 40 of the control system 100. In various embodiments, the control system 100 from the electrosurgical generator 10 may include one or more impedance loads 82 which are internal and/or integrated within the generator 10. It should be understood that the impedance loads 82 are not user-accessible and in various embodiments they are only used for the purpose of performing the self-verification system test of the generator 10. The one or more impedance loads 82, according to the embodiments of the present invention, may be resistive, capacitive, inductive, and any combination thereof. The self-verification system test process utilizes the impedance loads 82 to verify the voltage, current, power, and/or phase measurements of the generator 10.

In some embodiments, the impedance loads 82 are attached or integrated into the RF amplifier 40. In other embodiments, various configuration of the impedance loads 82 may be selected via one or more relays or switches 81. In one embodiment, the impedance loads 82 are in a parallel configuration. In another embodiment, the impedance loads 82 are in series, parallel or a combination thereof to provide different load configuration or values for other self-verification settings or testing.

The impedance loads 82, according to the embodiments of the present invention, are internal to avoid potential inaccuracies or errors due to the use of external impedance loads, such as connections, e.g., variable losses due to cabling, load properties, e.g., phase changes, user error, and equipment tolerances or errors thereto. The internal or integrated self-verification system including but not limited to the internal impedance loads 82 also avoids the need for additional measurement equipment, e.g., oscilloscopes, or specialized equipment, e.g., test electrosurgical instruments or keys, or accessories, e.g., adapters, along with any potential inaccuracies associated with the use of such equipment. Also, any setup time or scheduling of time to perform such verification is also avoided through the automatically scheduled and executed self-verification system test.

Table I summarizes exemplary self-verification processes performed in various RF regulation modes and RF resolution settings. In accordance with various embodiments, each process step may have specific regulation values and impedance loads configuration. After setting the regulation values and activating the verification relays 81, the RF amplifier 40 generates the RF output and supplies the RF output to the internal impedance loads 82 implemented within the RF amplifier 40 as directed by the self-verification system test. Accordingly, the control system 100 regulates the RF output to the set value and the feedback system 60 measures various electrical characteristics of RF output from the main channel 601, redundant channel 602 and verification channel 603 and feeds digitally the measured results to the microcontroller 50 for further processing. The primary microcontroller 50 compares the measured data or readings of the main and redundant channels 601 and 602 to the verification channel 603 and accordingly initiates or halts the supply of RF energy.

| | Voltage | Regulation | Regulation | | |
|---|---|---|---|---|---|
| Test | Mode | Unit | Value | | Load |
| 1 | High | Voltage | ~200 | V | Open |
| 2 | Normal | Voltage | ~80 | V | Open |
| 3 | Normal | Voltage | ~60 | V | Open |
| 4 | Normal | Voltage | ~60 | V | Resistor & Capacitor |
| 5 | Normal | Current | ~1 | A | Resistor & Capacitor |
| 6 | Normal | Power | ~80 | W | Resistor & Capacitor |
| 7 | Normal | Voltage | ~40 | V | Resistor |
| 8 | Normal | Current | ~1 | A | Resistor |
| 9 | Normal | Power | ~80 | W | Resistor |

| | | | | |
|---|---|---|---|---|
| 10 | Low | Passive - Limited | N/A | Resistor |

In accordance with various embodiments, in order for the self-verification tests to pass, the measurements of all the feedback system channels 601, 602 and 603 must match each other and/or be within a certain tolerance and/or match a target RF set point. More specifically, the main channel 601, redundant channel 602, and verification channel 603 have to be within a certain tolerance of a nominal value based on the internal load impedance configuration and target RF set point to prevent false positives if all readings of three channels are identical but offset. For example, if all the feedback system channels 601, 602 and 603 read or measure 20 volts from the RF output on a 60 volt test, the test will fail or not pass even though the channels identified identical voltage measurements.

In accordance with various embodiments, at the factory level after an electrosurgical generator 10 passes top level calibration and verification in the production process, initial verification channel offsets are calculated. The initial offset values are calculated by comparing the main and redundant channel readings to that of verification channel and offsetting them to the verification values. As a result, this sets the main, redundant, and verification channel readings to identical values. The primary microcontroller 50, in various embodiments, utilizing the ARM processor, applies these initial offsets internally for each verification test or process. Therefore, for example, if the internal impedance loads, e.g., shunt resistors, on any of the main, redundant and verification channels 601, 602 and 603 start to drift after the initial offset adjustment the self-verification system test of the generator 10 will identify the drift and/or otherwise indicate an error or failure.

A self-verification system test failure, according to the embodiments of the present invention, will display on the generator's LCD 14 and/or the generator 10 will not function such that no RF energy will be supplied to a connected electrosurgical instrument 20. In various embodiments, the generator's power can be cycled off and on and the self-verification system test will initiate again and, in various embodiments, after a predetermined number of failures in a particular sequence, e.g., two verification failures in a row, the electrosurgical generator 10 will fault or enter into a nonfunctional state and will require service before being able to operate, e.g., be able to supply RF energy to a connected electrosurgical instrument 20.

With the self-verification system operating at the generator or system power-on or startup, the electrosurgical generator 10 is configured to notify the surgeon of a potential generator issue, prior to any use of a connected electrosurgical instrument 20 or any supply of RF energy to the tissue or vessel through the electrosurgical instrument 20.

As such, without the self-verification system during the generator's startup or power-on operation, a conventional generator would not detect an RF output failure until a surgeon attempted to apply RF energy to tissue, if at all. The electrosurgical generator 10 according to the embodiments of the present invention is thus configured to detect failures on startup or a predetermined schedule which reduces troubleshooting and surgical operational time and avoids other potential surgical difficulties.

In various embodiments, the verification channel 603 has components of lower drift and higher resolution (higher quality and lower PPM/° C.). As such, the low thermal coefficients, which can dictate the drifts of the components, ensures that the verification channel 603 is more resistant to change over time and temperature. Additionally, components, such as shunt resistors, of the verification system channels 601, 602 and 603 may drift in value over time due to heat generated by the RF output. This drift can skew measurement values. However, since the components, e.g., the impedance loads, of the verification channel 603 are not utilized during normal RF output operations, the verification channel 603 is more resistant to drift.

As described further above, the verification channel 603 uses the normal RF output path, which is then redirected using relays 81 to self-verification resistors and capacitors, e.g., impedance loads 82, inside the electrosurgical generator 10 instead of the normal RF output path to a connected electrosurgical hand device 20. In various embodiments, high power loads, e.g., resistors, inductors or capacitors, are used to withstand or resist adverse effects, such as temperature, introduced through the application of RF energy. In one embodiment, high power resistors are provided and in other embodiments there are chassis mounted and placed in the airflow path of the generator to enhance heat dissipation.

In some embodiments, the impedance loads of the verification channel 603 are included or integrated on the same circuit board as the other channels 601 and 602 of the feedback system 60 that reduces the overall footprint of the electrosurgical generator 10 or the feedback system 60. In other embodiments, the impedance loads of the verification channel 603 are included or integrated on the different circuit board thereby reducing any affect to surrounding components due to heat generated by the impedance loads. In various embodiments, the impedance loads are varied, e.g., different value components, e.g., resistors, or different components, e.g., inductors used instead of capacitors for phase measurements, to enhance additional variations in the regulation modes being verified. In addition, the impedance loads may be in series, parallel or any combination thereof to provide various load configuration or values.

In some embodiments, the verification channel measurement circuit is positioned in series with the RF output circuit enhancing accuracy of the measurements and easing tracking or recording of such measurements. In other embodiments, the verification channel measurement circuit is separated, e.g., via a relay, from the RF output circuit and thus can enhance the resistant of the verification channel measurement circuit due to reduced operation time or the effects of heat due to supply of RF energy.

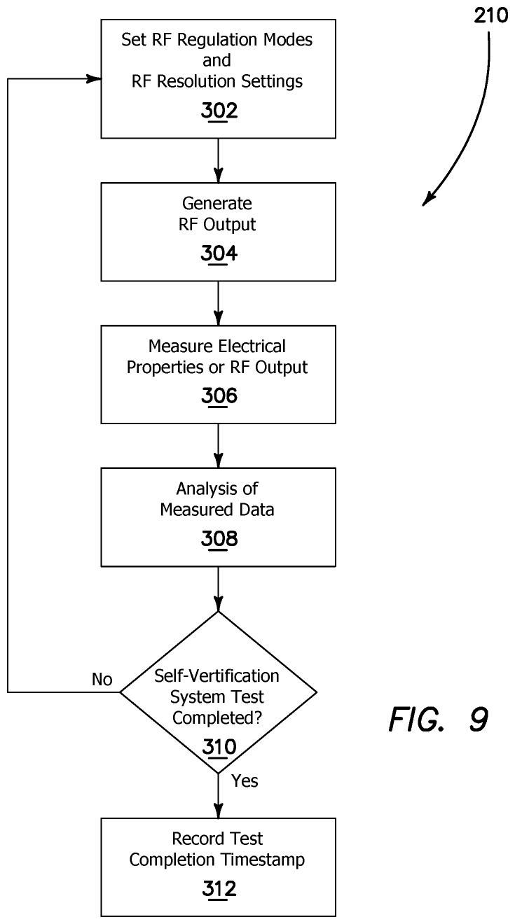

Referring next to FIG. 8, an embodiment of a self-verification system operations or processes according to the embodiments of the invention is shown. The depicted portion of the process 200 begins in step 202 where the algorithm powers on the electrosurgical generator 10 as a starting point. After starting-up the generator 10, a determination is made, at step 204, as to whether the previous self-verification system test has failed. If failure of the previous self-verification system test is detected, processing flows from block 204 to block 206 where the processing waits for a predetermined period of time (cooldown period) or power-on threshold, e.g., one minute, prior to initiating the self-verification system test at block 210. If failure of the previous self-verification system test is not detected, processing goes from block 204 to block 208 where another determination is made as to whether the predetermined period of time or power-on threshold has elapsed after the electrosurgical generator 10 is started.

If the power-on threshold is reached or exceeded, processing flows from block 208 to block 210 to initiate the self-verification system test. If the power-on threshold is not reached in block 208, processing goes to block 206 to wait for the predetermined period of time (cooldown period) or power-on threshold to pass. The processing then goes to block 210 for initiating the self-verification system test.

Once the self-verification system test is initiated at block 210, the electrosurgical generator 10 is verified using various processing tests. After completion of the self-verification system test, processing continues to block 212 where a determination is made as to whether a failure of the electrosurgical generator 10 has been detected during the self-verification process. If a failure is not detected, processing flows from block 212 to block 214 where the electrosurgical generator 10 is configured to supply RF energy to a connected electrosurgical hand device 20. In this embodiment, the electrosurgical generator 10 may provide a display or other indication that an electrosurgical device 20 can be connected to the generator 10 or the device already connected to the generator 10 is ready for surgical use. In other embodiments, device authentication and/or verification is also performed by the electrosurgical generator 10, prior to and/or after the generator 10 is verified by the self-verification system, before the connected electrosurgical instrument 20 is ready for surgical use. In accordance with various embodiments of the present invention, the processing determines that the electrosurgical generator 10 has no failure if one or more or all of the verification processes or tests are passed successfully.

If a failure of the electrosurgical generator 10 is detected, processing goes from block 212 to block 216 where another determination is made as to whether a failure threshold has been reached. The failure threshold determines if the self-verification system has failed consecutively a predetermined number of times, e.g., twice or more in a row. If the failure threshold is reached, processing flows from block 216 to block 218 where an error is generated causing the generator 10 to be inoperable. In this embodiment, the inoperative state of the generator 10 requires that the electrosurgical generator 10 to be serviced. In accordance with various embodiments of the present invention, the electrosurgical generator 10 notifies the user or surgeon, of the generator's error via an audible, tactile and/or visual indicator.

If the failure threshold is not reached in block 216, the processing then goes back to block 202 for restarting the electrosurgical generator 10 in which the processing can again attempt to verify the generator 10. This process continues until a failure of the electrosurgical generator 10 is not detected or the failure threshold is reached or exceeded.

With reference to FIG. 9, a flow diagram of an embodiment of process 210 for performing self-verification system test is shown. Self-verification process steps for various RF regulation modes and RF resolution settings are typically recorded or stored into a memory of the electrosurgical generator 10 of the present invention. In accordance with various embodiments, each process step may have specific regulation values and impedance loads configuration. Exemplary self-verification processes were provided further above in Table I. After initiating the self-verification system test, the depicted portion of the process begins in blocks 302 where the algorithm sets the RF regulation modes and RF resolution settings. As such, the processing sets the voltage mode, activates appropriate relays 81 for obtaining specific load configuration and sets the regulation value. The processing then goes to block 304 for generating RF output and providing RF energy to the verification system loads. In various embodiments, the RF amplifier 40 generates the RF output and supplies the RF output to internal loads 82 within the amplifier as directed by the self-verification system.

Once the RF output is generated, processing flows to block 306 where the feedback system 60 measures the electrical characteristics of the RF output. The control system 100, in accordance with various embodiments of the present invention, regulates the RF output to the set value as directed by the self-verification system and the feedback system 60 measures voltage, current, power, and/or phase from the main, redundant and verification channels 601, 602, and 603. After measuring the electrical characteristics of the RF output, processing flows to block 308 where the primary microcontroller 50 performs calculations, comparison and analysis of the measured data. In various embodiments, the feedback system 60 communicates the measured data and/or real and imaginary components thereof for the channels 601, 602, and 603 to the primary microcontroller 50 for further processing. The primary microcontroller 50 in various embodiments compares the data or readings of the main and redundant channels 601 and 602 to the verification channel 603 and if the main and redundant readings are less than a predefined tolerance or difference, e.g., 5%, the self-verification process step or test is passed or verified. Otherwise, the verification process step or test fails. In accordance with various embodiments of the present invention in both cases the results are recorded or stored into a memory of the electrosurgical generator 10 and/or the device key 21 of the electrosurgical instrument 20.

A determination is made at block 310 as to whether the self-verification system test of the electrosurgical generator 10 has been completed. If the self-verification system test is completed, the processing flows from block 310 to block 312 where a timestamp for recording test completion is generated before exiting the self-verification process. If the self-verification system test is not completed, the processing then goes back to block 302 for setting the new RF regulation mode and new RF resolution setting. This process continues until all of the self-verification process steps for verifying the electrosurgical generator 10 are performed. In various embodiments, the self-verification system test activates at a predetermined condition, such as on power-on, device connection and/or script or device activation, and/or along a predetermined schedule, e.g., at each predetermined conditions and/or a selected interval. In various embodiments, a separate button, switch or the like is provided on the generator, a connectable device and/or adapter utilized to activate the self-verification test.

The electrosurgical generator 10, according to the embodiments of the present invention, is configured to operate in one or more specific voltage modes. In various embodiments, the voltage modes are low, medium and high and in various embodiments the voltage mode adjusts or effects the feedback system 60. Voltage mode, in various embodiments, determines gain settings on the ADCs (Digital to Analog Convertors) used in the feedback system 60 and in various embodiments, high voltage is a maximum 300V gain settings, medium voltage is a maximum 150V gain settings, and low voltage is a maximum 10V gain settings. By having different gain settings for the different voltage modes, the resolution of the measurement of the feedback system 60 is increased.

According to the embodiments of the present invention, the RF amplifier may include an autotransformer directly connected to a primary transformer of the RF amplifer to supply high voltage mode. In various embodiments, when the RF amplifier 40 is operating in the high voltage mode (autotransformer on), the RF output may reach up to 300V and 4 A. On the other hand, when the autotransformer is off, the RF amplifier 40 may operate in the normal voltage mode, RF output limited to 150V and 8 A, and in the low voltage mode or passive mode which is limited to 10V and 100 mA.

In some embodiments, the self-verification system test varies the voltage modes to test or ensure the different gain settings are accurate across the main, redundant and verification channels 601, 602, and 603 of the feedback system 60. In other embodiments, different regulation modes of voltage, current and power are also included with the various voltage modes which are utilized and tested in the self-verification system test.

In accordance with various embodiments of the present invention, the measurements and calculations provided by the main and redundant channel 601 and 602 are compared to the measurements and calculations of the verification channel 603. If the channels 601, 602, and 603 all match and in various embodiments within a set tolerance, then the self-verification system test according to the embodiments of the present invention clears or allows the electrosurgical generator 10 to continue and thus allow RF output to a connected electrosurgical device 20. The main, redundant and verification channels 601, 602, and 603 in various embodiments must also be within a set tolerance compared to a nominal regulation value to also allow the verification system to clear the generator for operation.

According to the embodiments of the present invention, the low voltage mode is only used for passive measurement and does not set a constant voltage, current, or power. In various embodiments, it also does not utilize the verification channel 603 and thus RF output is provided in low voltage. In this embodiment, voltage and current are measured, the resistance is calculated and some or all of the measurements and calculations are compared to predefined values and/or within a predefined tolerance to further clear or allow the electrosurgical generator 10 for further operations.

In various embodiments, the self-verification system determines or checks the drift of the sense devices such as, for example, resistors, capacitors or inductors of the main and redundant channels 601 and 602 which are used to measure or calculate electrical properties of the RF output. If the drift of these components is minimal or within a predefined range and/or the unit can regulate to set values appropriately, then the RF output of the electrosurgical generator 10 is determined to have no failure by the self-verification system test and the self-verification process allows the generator 10 for further operational or surgical use.

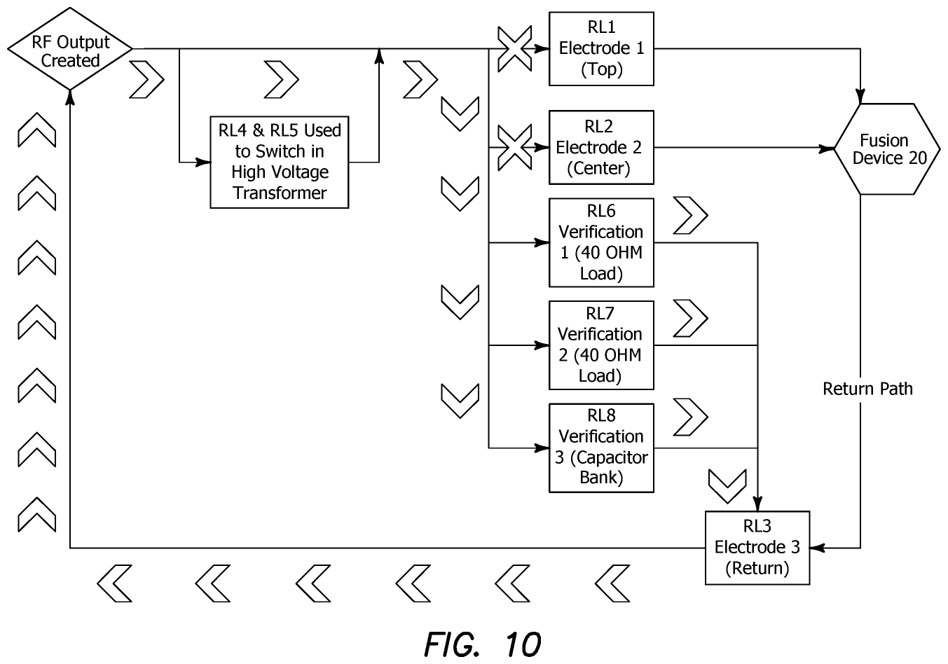

FIG. 10 illustrates a flow diagram of an example method for performing a self-verification system test of an electrosurgical generator 10. This process or system test verifies that the electrosurgical generator 10 can output RF energy at a set value, and that the feedback system 60 is accurately measuring this RF output across all channels 601, 602 and 603. In accordance with various embodiments of the present invention, RF output is first generated and thus provided to the self-verification system. In various embodiments, when the generator is powered on, e.g., the power button 18 on the front panel of the generator 10 is pressed, the generator 10 initiates the self-verification system process. This will cause the RF output to be supplied or be directed to an internal load rather than through a connected electrosurgical device 20. As shown in FIG. 10, the self-verification process bypasses or switches off relays 1 and 2 that would direct the RF output or the electrosurgical energy to a connected electrosurgical device, e.g., fusion device 20. It should be understood that an electrosurgical device does not need to be connected to the electrosurgical generator 10 for the self-verification system to operate or to its processes to proceed. Accordingly, the processing switches toward the internal loads as directed by the self-verification system.

As illustrated in FIG. 10, relays 6-8 are switched on/off in various sequences to provide the associated internal load configuration as needed. In various embodiments, relays 6 and 7 may be activated or used simultaneously and in parallel to assist in dissipating power and/or heat. On the other hand, relay 8 in various embodiments is switched on or used to include phase variations as desired. When a higher voltage RF output is needed, relays 4 and 5 of the exemplary embodiment are used to switch in a high voltage transformer. Relay 3, in various embodiments, is used or activated to provide the return path or complete the circuit for the self-verification system. In accordance with various embodiments of the present invention, the self-verification system may include a plurality of relays and loads including, for example, resistors, capacitors, inductors and various combinations thereof and as such other relays, loads and combinations thereof are not shown for ease of readability. In some embodiments, the self-verification system may include a timer to ensure that the self-verification system test is not activated or initiated needlessly or is activated as intended. As such, if the electrosurgical generator 10 is quickly powered on and off, e.g., in less than a second, the self-verification system does not activate and in various embodiments prevents potential thermal damage to the load such as, for example, resistors. In other embodiments, the timer is configured to start at or near power-on time and reaches or passes a predefined threshold to cause or initiate activation of the self-verification system.

The above description is provided to enable any person skilled in the art to make and use the electrosurgical devices or systems and perform the methods described herein and sets forth the best modes contemplated by the inventors of carrying out their inventions. Various modifications, however, will remain apparent to those skilled in the art. It is contemplated that these modifications are within the scope of the present disclosure. Different embodiments or aspects of such embodiments may be shown in various figures and described throughout the specification. However, it should be noted that although shown or described separately each embodiment and aspects thereof may be combined with one or more of the other embodiments and aspects thereof unless expressly stated otherwise. It is merely for easing readability of the specification that each combination is not expressly set forth.

Although the present invention has been described in certain specific aspects, many additional modifications and variations would be apparent to those skilled in the art. It is therefore to be understood that the present invention may be practiced otherwise than specifically described, including various changes in the size, shape and materials, without departing from the scope and spirit of the present invention. Thus, embodiments of the present invention should be considered in all respects as illustrative and not restrictive.

What is claimed is:

1. An electrosurgical system for performing surgical procedures comprising:
an electrosurgical generator adapted to perform a self-verification system test upon activation of the generator, the generator comprising:
a processor configured to:
cause initiating a self-verification system test after determining a predetermined period of time has elapsed after starting the generator;
cause supplying RF energy to a connected electrosurgical hand device, if a failure of the generator is not detected; and
cause generating a system error if the failure of the generator is detected and a failure threshold is reached; and
a plurality of impedance loads configured to perform the self-verification system test.

2. The electrosurgical system of claim 1 wherein the processor is further configured to cause restarting the generator to reinitiate the self-verification system test if the failure of the generator is detected and the failure threshold is not reached, and wherein the failure threshold comprises a number of consecutive times where the self-verification system test detects the failure of the generator.

3. The electrosurgical system of claim 1 wherein the self-verification system test comprises a verification algorithm verifying RF output of the generator across a plurality of RF regulation modes and a plurality of RF resolution settings, and wherein the processor is further configured to regulate the RF output of the generator to a predetermined set value for each of the plurality of RF regulation modes and RF regulation settings.

4. The electrosurgical system of claim 3 wherein the plurality of RF regulation modes comprises one of voltage, current, power and/or phase regulation mode, wherein the plurality of RF resolution settings comprises one of a low, medium or high voltage setting.

5. The electrosurgical system of claim 1 wherein the generator further comprising a feedback system measuring electrical properties of RF output across a plurality of channels, and wherein the processor allows for supplying RF energy to the connected electrosurgical hand device, if measurement values of the feedback system channels are matching each other and/or are within a certain tolerance across the plurality of RF regulation modes and RF resolution settings.

6. The electrosurgical system of claim 1 wherein the plurality of impedance loads are internal or integrated within the generator, wherein specific configurations of the plurality of impedance loads are attainable using a plurality of internal relays.

7. The electrosurgical system of claim 1 wherein upon activation of the generator, the processor is further configured to determine whether a failure of the previous self-verification system test has occurred, and wherein if the failure of the previous self-verification system test is detected, the processor cause the generator to wait for a cooldown period, and if the failure of the previous self-verification system test is not detected, the processor further determines whether a power-on threshold time has elapsed after starting the generator and prior to initiating the self-verification system test.

8. A method for performing an auto-verification and self-verification system of an electrosurgical generator prior to performing surgical procedure, the method comprising:
initiating a self-verification system test after determining a predetermined period of time has elapsed upon activation of the generator;
setting RF regulation modes and RF resolution settings after initiating the self-verification system test;
generating RF energy and directing RF output to a plurality of impedance loads within the generator;
measuring electrical characteristics of the RF output and analyzing the measured data;
determining whether the self-verification system test has been completed; and
recording self-verification system test completion timestamp upon completion of the self-verification system test.

9. The method of claim 8 further comprising the step of initiating supply of RF energy to a connected electrosurgical hand device, if a generator failure is not detected.

10. The method of claim 8 further comprising the step of generating a system error if a generator failure is detected and a failure threshold is reached, wherein the failure threshold is reached when the self-verification system test detects the generator failure for a number of consecutive times.

11. The method of claim 8 wherein the measuring step is performed by a feedback system of the generator; the measuring step comprises measuring the electrical properties of the RF output across a plurality of channels from the feedback system and communicating the real and imaginary components thereof for the plurality of channels to a microcontroller of the generator.

12. The method of claim 11 wherein the analyzing step comprises receiving the real and imaginary components of the measured data, performing power calculations and comparing the measurement values of the feedback system channels for determining whether a generator failure exist.

13. The method of claim 12 wherein the generator failure exist if measurements values of the feedback system channels are not matching each other and/or are not within a certain tolerance across the plurality of RF regulation modes and RF resolution settings.

14. The method of claim 12 wherein the microcontroller initiates or halts supply of RF energy to a connected electrosurgical hand device based on the comparison results.

15. An electrosurgical generator comprising:
a plurality of impedance loads integrated within an RF amplifier that supplies RF energy;
a feedback system measuring the electrical properties of RF energy directed to the plurality of impedance loads across a plurality of channels; and
a primary microcontroller configured to initiate a self-verification system test upon activation of the generator to verify the RF output of the generator across a plurality of RF regulation modes and RF resolution setting for determining whether a generator failure exist.

16. The electrosurgical generator of claim 15 wherein during the self-verification system test the primary microcontroller is configured to regulate the RF output of the generator to a predetermined set value for each of the plurality of RF regulation modes and RF regulation settings, wherein the plurality of RF regulation modes comprises one of voltage, current, power and/or phase regulation mode, and wherein the plurality of RF resolution settings comprises one of a low, medium or high voltage setting.

17. The electrosurgical generator of claim 15 wherein the generator failure exist if one or more or all measurements values of the feedback system channels are not matching each other and/or are not within a certain tolerance across the plurality of RF regulation modes and RF resolution settings.

18. The electrosurgical generator of claim 15 wherein the primary microcontroller is configured to receive the measured data from the feedback system, perform power calculations related thereto and compare the results from a main channel and a redundant channel of the feedback system to that of a verification channel from the feedback system.

19. The electrosurgical generator of claim 18 wherein, after completion of the self-verification system test, the microcontroller allows for supplying RF energy to a connected electrosurgical hand device, if the comparison results for the main and redundant channels are within a certain tolerance across a plurality of RF regulation modes and a plurality of RF resolution settings.

20. The electrosurgical generator of claim 18 wherein, after completion of the self-verification system test, the microcontroller allows for halting RF energy to the connected electrosurgical hand device, if the comparison results for the main and redundant channels are outside a certain tolerance for one of the plurality of RF regulation modes and RF resolution settings.
